(19) United States
(12) Patent Application Publication
(10) Pub. No.: US 2004/0204360 A1
(43) Pub. Date: Oct. 14, 2004

(54) Pharmaceutical composition intended for the treatment or for the prevention of diabetes, of cancer or of waardenburg syndrome

(76) Inventors: Rousseau Guy, Brussels (BE); Lemaigre Frederic, Waterloo (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/802,089

(22) Filed: Mar. 16, 2004

Related U.S. Application Data
(63) Continuation of application No. 09/763,535, filed on Feb. 20, 2001.
Application No. 09/763,535 is a 371 of international application No. PCT/BE99/00112, filed on Aug. 17, 1999.

(30) Foreign Application Priority Data
Aug. 17, 1998 (BE) 9800609

(51) Int. Cl. A61K 38/17; A61K 48/00

(52) U.S. Cl. 514/12; 514/44

(57) ABSTRACT

The invention concerns a pharmaceutical comprising an adequate pharmaceutical support and an element selected among the group consisting of a nucleotide sequence coding for a peptide of the ONECUT family a vector comprising said nucleotide sequence, the polypeptide sequence coded by said nucleotide sequence and/or a cell line transformed by said vector and expressing the peptide of the ONE CUT family.

### SUBJECT OF THE INVENTION

The present invention relates to a novel pharmaceutical composition intended for the treatment or for the prevention of diabetes or of cancer, in particular to a cellular therapy for diabetes by creating an artificial pancreas.

The present invention also relates to a diagnostic device intended for the diagnosis and for the monitoring of the progression of diabetes or of cancer.

### TECHNOLOGICAL BACKGROUND FORMING THE BASIS OF THE INVENTION

Diabetes is a generic term under which are designated disorders characterized by the combination of polyuria and polydipsia. Diabetes mellitus, also named hereinafter sugar diabetes, which can be type 1 or type 2 diabetes, is due to poor functioning of the beta cells of the endocrine pancreas (islets of Langerhans), which synthesize and secrete insulin (Gerich & Haeften, COED 5, pp. 144-148 (1998)). It is often accompanied (type 2 diabetes) by resistance of the target tissues to the action of the insulin.

Sugar diabetes is one of the most common metabolic diseases, in particular in the industrialized world (Leahy, COED 5, pp. 73-74 (1998)). It is characterized by a deficiency of the use of glucose, and can have serious and sometimes fatal pathological consequences, such as metabolic disorders, cardiovascular and neurological problems or retinal or renal lesions. Treatment with insulin requires one or more daily injections for life.

Consequently, there is a real need to replace these injections with transplantable systems (Gage et al., Nature 392, Supplement 3 (1998)).

### STATE OF THE ART

The document Lemaigre et al. (1996) describes a cDNA encoding hepatocyte nuclear factor 6, named hereinafter HNF-6. The naming of this molecule as hepatocyte nuclear factor (HNF) is an arbitrary naming which indicates that this molecule is a factor which is present in the nuclei of hepatocytes without prejudging whether or not it is related to the other molecules also identified as hepatocyte nuclear factor HNF-1 to HNF-4. This HNF-6 protein controls the transcription of certain genes in a small number of tissues in which it is expressed (Samadani & Costa (1996)). The expression of this molecule has in particular been identified in the mouse pancreas (Landry et al. (1997) and Rausa et al. (1997)).

It is also known that the HNF-6 molecule exerts a control on HNF-4 synthesis in cells in culture. However, none of these documents mentions that modification of the animal or human gene encoding HNF-6 is capable of causing diabetes in a whole organism.

Contrary to what is suggested in document WO 98/11254 and the publication by Duncan et al. (Science Vol. 282, pp. 692-695, July 1998), the HNF-3 molecule controls the synthesis of the HNF-4 molecule in cells in culture, but a modification affecting the gene encoding the HNF-3 molecule is not capable of causing diabetes in animals, including humans.

French patent application FR-2,696,755 describes an implantable capsule comprising an external case consisting of a hydrogel of acrylonitrile and of sodium methallylsulfonate and an internal core comprising an encapsulated substance which can consist of islets of Langerhans, of pancreatic beta cells or of hepatocytes. The case is a biocompatible membrane which is selectively permeable to insulin or to the nutrients required for the substance to be encapsulated. This product can be used in the transplantation of cells or of groups of cells such as islets of Langerhans, in order to overcome the insufficiency of insulin production in diabetic patients.

International patent application WO 95/09231 describes novel beta-insulin-secreting cell lines which can be in the form of “pseudo-islets” which can be encapsulated in a biocompatible hydrogel, which hydrogel is optionally incorporated into transplantable fibers intended to be introduced into the patient via a subcutaneous or intraperitoneal route in such a way as to treat insulin-dependent diseases.

International patent application WO 95/29988 describes a method for culturing cell lines, in particular pancreatic cells, capable of creating cellular islets which are reimplantable in vivo in mammals in such as way as to treat pancreatic diseases in humans or animals.

An essential characteristic of the cell lines which can be used in substitutive therapy for insulin-dependent diabetes is to be able to secrete insulin in response to glucose (glucose-stimulated insulin secretion, GSIS). This presumes that, in these cells, the expression of the genes involved in GSIS is stable. GSIS depends in particular on GLUT-2, which is the glucose transporter in beta cells, and on glucokinase, which is required for producing the GSIS signal from glucose. A recurrent problem of these lines is the loss of GLUT-2 and of glucokinase (Newgard et al.; (1997)). Another problem is apoptosis (Hohmeier et al., (1998)), cell death which can be caused by glucocorticoid hormones.

### AIMS OF THE INVENTION

The present invention is directed toward providing a novel pharmaceutical composition which is capable of being used in the prevention or treatment of diabetes or of cancer and which can be used either in the domain of genetic therapy or the domain of cell therapy in the form of cellular masses or the formation of an artificial pancreatic tissue or organ.

Another aim of the invention consists in providing a novel diagnostic device, such as a diagnostic kit intended to improve the diagnosis and/or monitoring of diabetes or of cancer, in particular to differentiate certain malignant progressions of the cancer.

### CHARACTERISTIC ELEMENTS OF THE INVENTION

The inventors have discovered, unexpectedly, that knocking out the HNF-6 gene in mice shows that this gene is essential for the functioning and formation of the islets of Langerhans and for the response of the organism to insulin. In addition, the inventors have shown that other proteins similar to HNF-6, which share with HNF-6 two particularities; on the one hand, the presence of a single cut domain and, on the other hand, the presence of the F48M50 dyad in the homeodomaine (Lannoy et al. (1998)), belonging to the same family named ONECUT (abbreviation OC) (Lannoy et al., (1998) and Jacquemin et al. (1999)), are also involved in some essential metabolic mechanisms. Among the family of proteins thus defined, which comprises in particular the HNF-6 protein, the OC-2 protein and the OC-3 protein, certain proteins have functions which are essential in animals, in particular in humans, especially in glucose metabolism. Mice in which the HNF-6 gene has been knocked out (hnf6−/− mice) have sugar diabetes. This is characterized by a GLUT-2 deficiency in the beta cells and by insufficient insulin secretion in response to glucose (Jacquemin et al., submitted for publication). The diabetes of the hnf6−/− mice is finally cured spontaneously, and this is accompanied by a large increase in OC-2 in the pancreas. These observations illustrate the importance of HNF-6 and of OC-2 in maintaining carbohydrate homeostasis, in particular through maintaining the differentiated phenotype of the beta cells. The inventors have shown, moreover, that HNF-6 can inhibit the effect of glucocorticoids (Pierreux et al. (1999)). In addition, such molecules may be used for treating, preventing or diagnosing the appearance and/or development of a certain number of disorders and of diseases, in particular diabetes or cancer, preferably melanoma.

The present invention therefore relates to a pharmaceutical composition comprising a suitable pharmaceutical vehicle and an element chosen from the group consisting of a nucleotide sequence encoding a protein which is a member of the ONECUT family, in particular the HNF-6, OC-2 or OC-3 molecules, the nucleotide and peptide sequences of which are described hereinafter, a vector comprising said nucleotide sequence, the encoded polypeptide sequence and/or a cell line transformed with said vector and expressing these said nucleotide sequences, which are in particular capable of encoding the HNF-6 protein or another member of the ONECUT family, such as the OC-2 or OC-3 molecules.

The expression “nucleotide sequence encoding HNF-6” is intended to mean, for the HNF-6, OC-2 or OC-3 protein, a nucleotide sequence the coding portions (included in the exons) of which correspond, respectively, to the coding sequence of the cDNA as already described by Lemaigre et al. (1996) or to the sequences as described below (encompassing the OC-2 and OC-3 sequences), and the sequences having more than 80%, preferably more than 85%, more particularly more than 90% or more than 95% homology (or sequence identity) with the sequence of the cDNA of the HNF-6 molecule as described by Lemaigre et al. (1996) or equivalent sequences capable of hybridizing with these nucleotide sequences (including the sequences of the OC-2 and OC-3 molecules). This hybridization takes place preferably under conditions which are sufficiently stringent so as to identify the various genomic sequences encoding an amino acid sequence identical or similar to the abovementioned sequences, in particular other sequences specific for other mammals, having the same function or being involved in the same biochemical mechanism, in particular those in the examples below, but possibly different (in particular due to the redundancy of the genetic code). Stringent hybridization conditions are, in particular, as follows: hybridization at 40° C. in 50% of formamide, 5×SSC 20 mM sodium phosphate, pH 6.8, washing in 0.2×SSC at 50° C. Modifications of these conditions can be provided by those skilled in the art as a function of the length and of the GC-nucleotide content in the sequence to be hybridized. Other hybridization conditions are in particular those described by Sambrook et al., §§ 9.47-9.51 in Molecular Cloning: A Laboratory Manual, Cold Spring Harbor, Laboratory Press, Cold Spring Harbor, N.Y. (1989)).

According to the invention, the gene encoding the HNF-6 used concerns genomic sequences encoding both the alpha and beta isoforms of HNF-6, as described by Lannoy et al. (1998).

The pharmaceutical composition of the invention can be used to produce genetic and/or cell therapy for a patient likely to develop diabetes or suffering from diabetes, or likely to develop a cancer or suffering from a cancer, in particular from a melanoma. In the domain of genetic therapy, the nucleotide sequence of the invention can be administered to the patient or to cell lines from the patient via ex vivo treatment in naked form using methods well known to the person skilled in the art or via a vector, preferably chosen from the group consisting of plasmids, viruses, phagemids, lipid vesicles such as cationic lipids, liposomes or a mixture of these. The vector will incorporate all the elements required to obtain the expression of the nucleotide sequence according to the invention in the patient, preferably in the specific cell lines to be treated, such as the pancreatic cells involved in insulin synthesis, the hepatic cells involved in insulin response or cells of the epidermis or of the dermis which are likely to develop a melanoma.

The pharmaceutical composition of the Inventor can also be used in cell therapy by direct injection of the cells using an in vivo or ex vivo method or by forming an artificial cellular aggregate as described in patent applications FR-2,696,755, WO 95/09231 and WO 95/29988. Proliferation of the cells transformed with the nucleotide sequence of the invention or the vector of the invention can be obtained using methods well known to those skilled in the art, in particular those described in patent applications WO 97/49728 and WO 95/29988.

The pharmaceutical vehicle according to the invention varies according to the method of administration chosen (intravenous, intramuscular, oral, etc.) and is an excipient well known to those skilled in the art, in the form of tablets, pills, capsules, solutions, syrups, etc. This component optionally comprises adjuvants (in particular a growth hormone) well known to those skilled in the art, so as to induce synergistic effects or to suppress certain specific immune or cellular reactions, or so as to reduce certain unwanted side effects or toxic effects of the active principle or of the vehicle of the invention.

The percentage of active product (nucleotide sequence, amino acid sequence or fragments thereof, vector, cell line, etc.) in the pharmaceutical composition can vary according to very wide ranges which are limited only by the frequency of administration, the tolerance and the level of acceptance of the composition according to the invention by the patient.

The present invention also relates to the use of the pharmaceutical composition of the invention for preparing a medicinal product intended for the treatment and/or for the prevention of type 1 or type 2 diabetes and of the disorders linked to diabetes, in particular of the disorders linked to the poor functioning of the beta cells of the endocrine pancreas which synthesize and secrete insulin, and/or for the treatment of cancer, in particular of melanoma.

Another aspect of the present invention relates to the method for treating a patient, in particular a patient likely to develop diabetes, suffering from diabetes, likely to develop a cancer or suffering from a cancer, especially a melanoma, by which the pharmaceutical composition of the invention is administered to said patient using an in vivo or ex vivo treatment method.

A final aspect of the present invention relates to the protection, as a novel product, of the nucleotide and peptide sequences encoding the OC-2 molecule, and the sequences homologous to the OC-2 and OC-3 sequences. The expression “homologous sequence” is intended to mean the genetic sequences having more than 80%, preferably more than 85%, more particularly more than 90% or more than 95% homology (or sequence identity) with the nucleic acid and amino acid sequences as described in the appended sequence listing (SEQ ID No. 1 to SEQ ID No. 4), provided that this sequence does not encompass the sequence of the HNF-6 molecule as described by Lemaigre et al. (1996).

Homologous sequences are also defined as sequences which can hybridize with the nucleotide sequences SEQ ID No. 1 and SEQ ID No. 3 encoding the OC-2 and OC-3 molecules. This hybridization takes place preferably under sufficiently stringent conditions, as described above.

Besides the therapeutic and prophylactic application mentioned above, a second application of these novel sequences encoding the OC-2 and OC-3 molecules is their use in the domain of the diagnosis and/or of the monitoring of various disorders, in particular of diabetes and/or of cancer.

A final aspect of the present invention therefore relates to a diagnostic device such as a diagnostic kit comprising said nucleotide and/or peptide sequences of the OC-2, OC-3 and HNF-6 sequences, and the various reagents intended for the diagnosis and for the monitoring of diseases, in particular of diabetes, of cancer, in particular the diagnosis and monitoring of the progression of melanoma for detection based on the technical methods chosen from the group consisting of in situ hybridization, hybridization and identification with labeled antibodies, in particular by the ELISA or RIA technique, methods of hybridization on a filter, on a solid support, in solution, in sandwich or on a gel by dot blot hybridization or by Northern blot, Southern blot or Western blot hybridization, by labeling with isotopes or without isotopes (such as immuno-fluorescence or biotin labeling), by the so-called cold probe technique or by genetic amplification (or in particular using PCR, RT-PCR, LCR or CPR amplifications), of double immunodiffusion, of counter-immunoelectrophoresis, of hemagglutination or other techniques well known to those skilled in the art which allow specific identification of nucleotide and/or protein sequences.

The diagnostic device can also comprise elements which allow optional purification of a sample obtained from a human or animal body (such as a physiological liquid), prior treatment of this sample and option preamplification of this sample, as well as diagnosis and quantification of this possible nucleotide or protein sequence and an analysis correlated with the general condition of the animal or human patient treated. These various steps can be carried out manually or using an automatic machine.

The present invention will be described in detail in the nonlimiting examples given below with reference to the appended figures.

### EXAMPLE 1

### Detection of Melanocyte Differentiation

The function of the melanocytes of the skin, in response to irradiation by UV radiation (Carreira), is to protect the keratinocytes against damage to the DNA induced by UV radiation, via the production of the pigment melanin. More than 70 genes which affect melanocyte development have been identified by genetic analysis, and more than 20 of them have been cloned (Opdecamp (1997)).

A transcription factor (microphthalmia-associated transcription factor (MITF)) is involved in melanocyte differentiation in humans (Tachibana (1996)). It is known that mutations in the MITF gene are associated with Waardenburg syndrome (Tachibana (1994)). It is also known that mutants of the Pax-3 or CREB transcription factors which do not have this transcription activity are associated with the type 1 and type 3 Waardenburg syndromes (Tassabehji). Since the Pax-3 gene encodes an MITF-activating molecule, the identification of other transcriptional factors which affect the MITF gene contribute to improving the diagnosis and monitoring of cancerous diseases and can find applications in the treatment and/or prevention of these diseases, in particular of the Waardenburg syndrome.

### Experimental Procedure/Reverse Transcription

A reverse transcription PCR (RT-PCR) is carried out in order to detect the expression of the human mRNAs of the OC-2 and HNF-6 molecules in melanocytes and in various melanoma cells, one microgram of total RNA being reverse [transribed by] using the murine Moloney leukemia virus reverse transcriptase and other reagents (random hexamers (Life technology Inc.)). The cDNAs of these OC-2 and HNF-6 molecules were amplified by PCR and the specificity of the amplified products was identified by Southern blotting experiments, as described by Jacquemin et al. (1997-1999).

The integrity of the RNA preparations was controlled by amplification of a beta-actin cDNA fragment. The negative controls including the RT-PCR were carried out in the absence of reverse transcriptase.

### Melanocyte Cell Lines

The 397-MEL and 526-MEL cell lines were obtained from the National Cancer Institute KANG.

The LB373-MEL, BB74-MEL and LB1622-MEL cell lines were obtained from the Ludwig Institute of Cancer Research, Brussels, Belgium).

### Expression of the Genes Encoding the OC-2 Molecule in Melanocytes

The ONECUT proteins are in particular expressed in the cells of human skin. However, the levels of mRNA encoding the OC-2 molecule are particularly high. The expression of mRNA encoding the HNF-6 molecule is low in this tissue (Jacquemin et al. (1999)).

In order to identify a cell type expressing the OC-2 molecule, the inventors carried out an RT-PCR analysis of the RNA of melanocytes and of melanomas.

The PCR products were subjected to analysis by Southern blotting based on the use of radioactive probes.

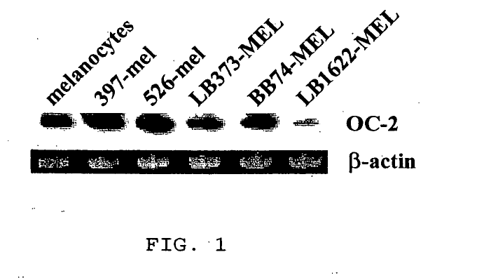

The results given in FIG. 1 show that it is possible to use the nucleotide sequences of the ONECUT family in order to obtain a differentiated diagnosis of the development of the melanoma.

These results show that only the OC-2 gene is expressed in the melanocytes of the skin.

By contrast, the two genes are highly expressed in various melanoma cell lines. These two genes are expressed at similar levels in the melanoma lines, but the general expression varies depending on the cell lines tested.

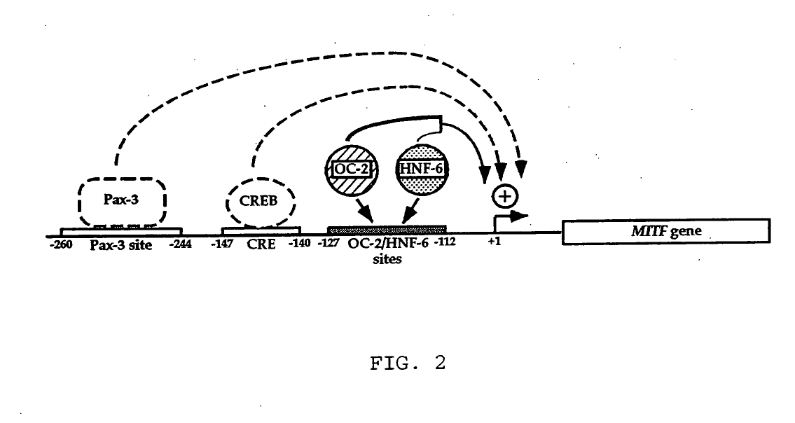

Additional assays of transfection of cell lines with plasmid constructs made it possible to demonstrate that the proximal ONECUT binding site of the MITF promoter is important for the activation of this promoter and that the OC-2 and HNF-6 transcription factors can stimulate the MITF promoter (see FIG. 2). Through the discovery that the HNF-6 transcription factor is expressed in melanocytes, it appears that the OC-2 transactivation factor is responsible for the stimulation of the MITF promoter in this type of cell line, and therefore involved in melanocyte development. Given that the HNF-6 gene is expressed at particularly early stages of melanocyte differentiation, and that it is also identified in the melanoma cell lines, it may be considered that HNF-6 is a melanoma cell marker and makes it possible, therefore, to distinguish these cells from already differentiated melanocytes.

Consequently, the genetic sequences of the invention can be advantageously used for improving and adding to the diagnosis and monitoring of various infections and pathologies, in particular of certain types of cancer (such as melanoma), and other syndromes, in particular Waardenburg syndrome, which involves modified expression of the human MITF genes which affect in particular the abnormal development of the melanocytes present in the skin, in the ears and in the eyes.

The gene encoding the OC-2 factor is also a suitable candidate in the domain of genetic therapy for controlling melanocyte development or for treating Waardenburg syndrome.

### EXAMPLE 2

### Cell Therapy of a Patient

The operating protocol described below can be applied to patients suffering from various pathologies, in particular patients likely to develop diabetes or suffering from diabetes, or likely to develop or suffering from a cancer, in particular a melanoma, or patients affected by Waardenburg syndrome.

It is clearly understood that the pharmaceutical composition of the invention, which is based on a genetic or cell therapy, can also be combined with treatments based on the use of other gene-regulating systems, in particular based on the use of the Pax-3 or CREB transactivation factors previously described (see FIG. 2).

Briefly, the treatment consists in implanting, in a diabetic animal, a line of cells which will have been programmed for GSIS by stable transfection of HNF-6 (or of OC-2). As described above, HNF-6 is, in fact, reputed to maintain in cells the expression of the genes of the differentiated phenotype, in particular GLUT-2 and glucokinase in beta cells, and HNF-6 might oppose the apoptotic effect of glucocorticoids on the implanted cells.

Rats (Wistar males weighing 200-250 g) are made diabetic with a single intravenous injection of streptozotocin (55 mg/kg). After two weeks, to confirm that diabetes has set in, the glucose in the urine is assayed (>15 mM by the Ames “strip” test). These rats received, by intraperitoneal injection, 10 or 50 microspheres (800-900 microns in diameter), each containing 200 000 cells of the “test” line. These microspheres, described by Kessler et al. (1992) (see also French patent application FR-2,696,755), are permeable to insulin, which must be able to exit therefrom, and to the signals of GSIS (such as glucose), which must be able to enter therein. They are impermeable to the agents of rejection by the immune system, but not to glucocorticoids. The “test” line is the RIN 1046-38 line, obtained from a rat insulinoma and cultured according to Clark et al. (1990). Stable transfectants either of HNF-6 or of OC-2 are obtained by electroporating the RIN 1046-38 cells with a plasmid vector comprising a bacterial origin of replication, an ampicillin resistance gene, the DNA complementary to OC-2 or to HNF-6 under the control of the cytomegalovirus promoter/enhancer and a complementary DNA encoding neomycin phosphotransferase. The complementary DNA encoding neomycin phosphotransferase is cloned 3′ of an internal ribosome entry site, itself located 3′ of the DNA complementary to HNF-6 or OC-2, such that the cytomegalovirus promoter controls the synthesis of a single bicistronic RNA encoding both HNF-6 or OC-2 and neomycin phosphotransferase. A polyadenylation signal, derived from the SV40 virus and located 3′ of the complementary DNA encoding neomycin phosphotransferase, ensures the polyadenylation of the bicistronic RNA. After transfection of the RIN 1046-38 cells according to Clark et al. (1997), the stable transfectants are selected by treatment with geneticin (500 μg/ml) for two weeks.

This method can be transposed to insulin-dependent (decompensated type II or type I diabetes) humans as described in Aebischer et al. (1999).

### References

Aebischer, O. et al., Nature Medicine 5, p. 852 (1999)

Carreira, S. et al., Mol. Cell. Biol. 18, pp. 5099-5108 (1998)

Clark, S. A. et al., Endocrinology 127, pp. 2779-2788 (1990)

Clark, S. A. et al., Diabetes 46, pp. 958-967 (1997)

Hohmeier, H-E. et al., J. Clin. Invest. 101, pp. 1811-1820 (1998)

Jacquemin et al., J. Biol. Chem. 272, pp. 12928-12937 (1997)

Jacquemin et al., J. Biol. Chem. 274, pp. 2665-2671 (1999)

Kahn, B. B., Cell 92, pp 593-596 (1998)

Kessler, L. et al., Biomaterials 13, pp. 44-49 (1992)

Landry, C. et al., Dev. Biol. 192, pp. 247-257 (1997)

Lannoy, V. J. et al., J. Biol. Chem. 273, pp. 13552-13562 (1998)

Lemaigre, F. P. et al., Proc. Natl. Acad. Sci. USA. 93, pp. 9460-9464 (1996)

Newgard, C. B. et al., Diabetologia 40, pp. S42-S47 (1997)

Opdecamp, K. et al., Development 124, pp. 2377-2386 (1997)

Pierreux, C. E., et al., Proc. Natl. Acad. Sci. (USA) 96, pp. 8961-8966 (1999)

Rausa, F. et al., Dev. Biol. 192, pp. 228-246 (1997)

Samadani, U. & Costa, R. H., Mol. Cell. Biol. 16, pp. 6273-6284 (1996)

Tachibana, M. et al., Human Mol. Genet. 3, pp. 553-557 (1994)

Tachibana, M. et al., Nature Genet. 14, pp. 50-54 (1996)

Tassabehji, M. et al., Nature 355, pp. 635-636 (1992)

1. A pharmaceutical composition comprising An acceptable pharmaceutical vehicle and an element chosen from the group consisting of a nucleotide sequence encoding a protein of the ONECUT family characterized by the presence of a single CUT domain and the presence of an F48M50 dyad in the homeo domain, a vector comprising this nucleotide sequence, the protein sequence encoded by this nucleotide sequence and/or a cell line transformed with said vector and expressing said protein of the ONECUT family.

2. The pharmaceutical composition as claimed in claim 1, characterized in that the protein of the ONECUT family is HNF-6 in its two isoforms.

3. The pharmaceutical composition as claimed in claim 1, characterized in that the protein of the ONECUT family is OC-2, the amino acid sequence of which is SEQ ID No. 2.

4. The cellular pharmaceutical composition as claimed in claim 1, characterized in that the protein of the ONECUT family is OC-3, the amino acid sequence of which is SEQ ID No 3.

5. The pharmaceutical composition as claimed in any one of the preceding claims, characterized in that said nucleotide and polypeptide sequences are human nucleotide and polypeptide sequences.

6. The pharmaceutical composition as claimed in any one of the preceding claims, characterized in that the vector is chosen from the group consisting of plasmids, viruses, phagemids, lipid vesicles, in particular cationic vesicles, liposomes or a mixture of these.

7. The use of the pharmaceutical composition as claimed in any one of the preceding claims, for preparing a medicinal product intended for the prevention and/or for the treatment of type 1 or type 2 diabetes or of disorders linked to diabetes, for the prevention and/or for the treatment of cancer, in particular of melanoma, and for the prevention and for the treatment of Waardenburg syndrome.

8. A method of therapeutic treatment of a patient, preferably of a human patient, likely to develop or suffering from diabetes, from a cancer, in particular from a melanoma, or from Waardenburg syndrome, characterized in that the pharmaceutical composition as claimed in any one of claims 1 to 4 is administered ex vivo by isolating a body fluid or one or more cells from the patient, treating said cells or the cells present in this body fluid with the pharmaceutical composition of the invention or with the vector included in this pharmaceutical composition, and reinjecting into said patient the transformed cells.


SEQUENCE LISTING (4 sequences)

SEQ ID NO: 1 - DNA, 1655 bp, Homo sapiens
gcccccgcccgccccgggccctgatggactgaatgaaggctgcctacaccgcctatcgatgcctcaccaaagacctagaacgtgcgccatgaacccggagctgacaatggaaagtctgggcactttgcacggcgcgcgcggcggcggcagtggcgggggcggcggcgggggcggcgggggcggcggcgggggcccgggccatgagcaggagctgctggccagccccagcccccaccacgcgcgccgcggcccgcgtggctcgctgcggggccctccgccgcctccaaccgcgcaccaggagctgggcacggcggcagcggcggcagcggcggcgtcgcgctcggccatggtcaccagcatggcctcgatcctggacggcggcgactaccggcccgagctctccatcccgctgcaccacgccatgagcatgtcctgcgactcgtctccgcctggcatgggcatgagcaacacctacaccacgctgacaccgctccagccgctgccacccatctccaccgtgtctgacaagttccaccaccctcacccgcaccaccatccgcaccaccaccaccaccaccaccaccagcgcctgtccggcaacgtcagcggcagcttcaccctcatgcgcgacgagcgcgggctcccggccatgaacaacctctacagtccctacaaggagatgcccggcatgagccagagcctgtccccgctggccgccacgccgctgggcaacgggctaggcggcctccacaacgcgcagcagagtctgcccaactacggtccgccgggccacgacaaaatgctcagccccaacttcgacgcgcaccacactgccatgctgacccgcggtgagcaacacctgtcccgcggcctgggcaccccacctgcggccatgatgtcgcacctgaacggcctgcaccacccgggccacactcagtctcacgggccggtgctggcacccagtcgcgagcggccaccctcgtcctcatcgggctcgcaggtggccacgtcgggccagctggaagaaatcaacaccaaagaggtggcccagcgcatcacagcggagctgaagcgctacagtatcccccaggcgatctttgcgcagagggtgctgtgccggtctcaggggactctctccgacctgctccggaatccaaaaccgtggagtaaactcaaatctggcagggagaccttccgcaggatgtggaagtggcttcaggagcccgagttccagcgcatgtccgccttacgcctggcagcgtgcaaacgcaaagagcaagaaccaaacaaagacaggaacaattcccagaagaagtcccgcctggtgttcactgacctccaacgccgaacactcttcgccatcttcaaggagaacaaacgcccgtcaaaggagatgcagatcaccatttcccagcagctgggcctggagctcacaaccgtcagcaacttcttcatgaacgcccggcgccgcagcctggagaagtggcaagacgatctgagcacagggggctcctcgtccacctccagcacgtgtaccaaagcatgatggaaggactctcacttgggcacaagtcacctccaaatgaggacaacagataccaaaagaaaacaaaggaaaaagacaccggattcctagctggggcccttcactggtg

SEQ ID NO: 2 - protein, 485 aa, Homo sapiens
MNPELTMESLGTLHGARGGGSGGGGGGGGGGGGGGPGHEQELLASPSPHHARRGPRGSLRGPPPPPTAHQELGTAAAAAAAASRSAMVTSMASILDGGDYRPELSIPLHHAMSMSCDSSPPGMGMSNTYTTLTPLQPLPPISTVSDKFHHPHPHHHPHHHHHHHHQRLSGNVSGSFTLMRDERGLPAMNNLYSPYKEMPGMSQSLSPLAATPLGNGLGGLHNAQQSLPNYGPPGHDKMLSPNFDAHHTAMLTRGEQHLSRGLGTPPAAMMSHLNGLHHPGHTQSHGPVLAPSRERPPSSSSGSQVATSGQLEEINTKEVAQRITAELKRYSIPQAIFAQRVLCRSQGTLSDLLRNPKPWSKLKSGRETFRRMWKWLQEPEFQRMSALRLAACKRKEQEPNKDRNNSQKKSRLVFTDLQRRTLFAIFKENKRPSKEMQITISQQLGLELTTVSNFFMNARRRSLEKWQDDLSTGGSSSTSSTCTKA

SEQ ID NO: 3 - DNA, 1485 bp, Homo sapiens
atggagctgagcctggagagcctggggggcctgcacagcgtggcccacgcgcaggcgggcgagctgctgagcccgggccacgcgcgctcggcggcggcgcagcaccgcggcctggtggcgcccgggcgcccgggcctggtggccggcatggcgagcctgctggacggcggcggcggcggcggcggtgggggcgccgggggcgcgggcggcgcgggcagcgcgggcggcggcgcggacttccgcggggaactggcgggcccgctgcacccggcaatgggcatggcctgcgaggcgccgggcctgggcggcacctacacgacgctcacgcccctgcagcacctgccgccgctcgcggccgtggccgacaagttccaccagcacgcggcggccgcggccgtggccggggcgcacggcggccatccccacgcgcacccgcacccggcggccgcgccgcccccgccacccccgccgcagcgtctggcggccagcgtgagcggcagcttcaccctcatgcgcgacgagcgggcggcgctcgcctccgtgggccacctctacggaccctacggcaaggagctgcccgccatggggtcgccgctgtcgccgctgcccaacgcgctgccgcccgcgctgcacggcgccccgcagcccccgccgccgccaccacccccgccgctggccgcctacggcccgccaggccacctggctggggacaagctgctgccgcccgccgccttcgagccgcacgccgcgctgctgggacgcgcggaggacgcactggcccgcgggctgcccggaggcggcggcggcacaggcagcggcggagcgggcagcgggagcgccgcggggctgctggcgccgctgggcgggctggcggcggccggggcgcacgggccgcacgggggaggcggcggccccggcgggagcggcggcggccccagcgcgggcgcagcggccgaggagatcaacaccaaggaggtggcgcagcgcatcacggcggagctgaagcgctacagcatcccgcaggcaatcttcgcgcagcggatcctgtgtcgctctcagggcacgctctccgacctgctgcgcaaccccaagccgtggagcaagctcaaatccggccgcgagaccttccgcaggatgtggaagtggctgcaggagccagagttccagcgcatgtcggcgctgcgcttggcagcgtgcaagcgcaaggaacaggagcagcagaaggagcgcgccctgcagcccaagaagcagcgcctggtgttcaccgacctgcagcgacgcacgctgatcgccatcttcaaggagaacaagcggccgtccaaggagatgcaggtcaccatctcgcagcagctcggcttggagctcaacaccgtcagcaacttcttcatgaacgcgcggcgccgctgcatgaaccgctgggctgaggagcccagcacggcccccgggggccccgccggcgccacggccactttctccaaggcctga

SEQ ID NO: 4 - protein, 494 aa, Homo sapiens
MELSLESLGGLHSVAHAQAGELLSPGHARSAAAQHRGLVAPGRPGLVAGMASLLDGGGGGGGGGAGGAGGAGSAGGGADFRGELAGPLHPAMGMACEAPGLGGTYTTLTPLQHLPPLAAVADKFHQHAAAAAVAGAHGGHPHAHPHPAAAPPPPPPPQRLAASVSGSFTLMRDERAALASVGHLYGPYGKELPAMGSPLSPLPNALPPALHGAPQPPPPPPPPPLAAYGPPGHLAGDKLLPPAAFEPHAALLGRAEDALARGLPGGGGGTGSGGAGSGSAAGLLAPLGGLAAAGAHGPHGGGGGPGGSGGGPSAGAAAEEINTKEVAQRITAELKRYSIPQAIFAQRILCRSQGTLSDLLRNPKPWSKLKSGRETFRRMWKWLQEPEFQRMSALRLAACKRKEQEQQKERALQPKKQRLVFTDLQRRTLIAIFKENKRPSKEMQVTISQQLGLELNTVSNFFMNARRRCMNRWAEEPSTAPGGPAGATATFSKA